(19) United States
(12) Patent Application Publication
(10) Pub. No.: US 2021/0173024 A1
(43) Pub. Date: Jun. 10, 2021

(54) SWAGED COMPONENT MAGNET ASSEMBLY FOR MAGNETIC RESONANCE IMAGING

(71) Applicant: Hyperfine Research, Inc., Guilford, CT (US)

(72) Inventor: Cedric Hugon, Guilford, CT (US)

(21) Appl. No.: 17/116,680

(22) Filed: Dec. 9, 2020

Related U.S. Application Data
(60) Provisional application No. 62/946,075, filed on Dec. 10, 2019.

(51) Int. Cl. G01R 33/38; G01R 33/44; G01R 33/3873; A61B 5/055

(52) U.S. Cl. CPC G01R 33/3802; A61B 5/055; G01R 33/3873; G01R 33/445

(57) ABSTRACT

Systems and methods for providing a B₀ magnetic field for a magnetic resonance imaging (MRI) system using swaged permanent magnet components are provided. A first apparatus comprises a cylindrical shell forming a bore extending along a common longitudinal direction, the cylindrical shell comprising: a first plurality of ferromagnetic rings including a first ferromagnetic ring with an angularly varying magnetization orientation, and a second plurality of rings. A second apparatus comprises ferromagnetic rings including a first ferromagnetic ring and a second ferromagnetic ring, the first ferromagnetic ring having a first magnetization and the second ferromagnetic ring having a second magnetization, wherein: the first magnetization and the second magnetization have first radial and axial components and second radial and axial components, respectively; and the first radial and axial components are different than the second radial and axial components.

### CROSS REFERENCE TO RELATED APPLICATIONS

This application claims priority under 35 U.S.C. § 119(e) to U.S. Provisional Patent Application Ser. No. 62/946,075, titled “SWAGED COMPONENT MAGNET ASSEMBLY FOR MAGNETIC RESONANCE IMAGING,” filed on Dec. 10, 2019, which is incorporated by reference in its entirety herein.

### BACKGROUND

Magnetic resonance imaging (MRI) provides an important imaging modality for numerous applications and is widely utilized in clinical and research settings to produce images of the inside of the human body. As a generality, MRI is based on detecting magnetic resonance (MR) signals, which are electromagnetic waves emitted by atoms in response to state changes resulting from applied electromagnetic fields. For example, nuclear magnetic resonance (NMR) techniques involve detecting MR signals emitted from the nuclei of excited atoms upon the re-alignment or relaxation of the nuclear spin of atoms in an object being imaged (e.g., atoms in the tissue of the human body). Detected MR signals may be processed to produce images, which in the context of medical applications, allows for the investigation of internal structures and/or biological processes within the body for diagnostic, therapeutic and/or research purposes.

### SUMMARY

Some embodiments are directed to an apparatus for providing a B₀ magnetic field for a magnetic resonance imaging (MRI) system. The apparatus comprises a cylindrical shell forming a bore extending along a common longitudinal direction. The cylindrical shell comprises a first plurality of ferromagnetic rings including a first ferromagnetic ring with an angularly varying magnetization orientation and a second plurality of rings.

Some embodiments are directed to a method of manufacturing an apparatus for providing a B₀ magnetic field for a magnetic resonance imaging (MRI) system. The method comprises manufacturing a ferromagnetic cylindrical shell at least in part by: placing a magnetic metal alloy powder in an annular volume between an outer cylindrical tube and an inner cylindrical tube; applying a magnetic field to the magnetic metal alloy powder while compressing the magnetic metal alloy powder; bonding the magnetic metal alloy powder to form at least a part of the ferromagnetic cylindrical shell; magnetizing the ferromagnetic cylindrical shell to have an angularly varying magnetization orientation; partitioning the ferromagnetic cylindrical shell into a first plurality of ferromagnetic rings; and assembling, from the first plurality of ferromagnetic rings and a second plurality of rings, a cylindrical shell forming a bore extending along a common longitudinal direction.

Some embodiments are directed to a method of manufacturing an apparatus for providing a B₀ magnetic field for a magnetic resonance imaging (MRI) system. The method comprises manufacturing a ferromagnetic cylindrical shell at least in part by: placing magnetic metal alloy powder and non-ferromagnetic powder in an annular volume between an outer cylindrical tube and an inner cylindrical tube; applying a magnetic field to the magnetic metal alloy powder while compressing the magnetic metal alloy powder; bonding the magnetic metal alloy powder and the non-ferromagnetic powder to form the ferromagnetic cylindrical shell; and magnetizing the ferromagnetic cylindrical shell to have an angularly varying magnetization orientation.

Some embodiments are directed to a method of manufacturing an apparatus for providing a B₀ magnetic field for a magnetic resonance imaging (MRI) system. The method comprises manufacturing a ferromagnetic cylindrical shell at least in part by: placing magnetic metal alloy powder in an annular volume between an outer cylindrical tube and an inner cylindrical tube; applying a magnetic field to the magnetic metal alloy powder while compressing the magnetic metal alloy powder; bonding the magnetic metal alloy powder and the non-ferromagnetic powder to form the ferromagnetic cylindrical shell; selectively magnetizing first ring regions of the ferromagnetic cylindrical shell to have a first angularly varying magnetization orientation; and selectively magnetizing second ring regions of the ferromagnetic cylindrical shell to have a second angularly varying magnetization orientation, the second angularly varying magnetization orientation varying in a direction opposing that of the first angularly varying magnetization orientation.

Some embodiments are directed to an apparatus for providing a B₀ magnetic field for a magnetic resonance imaging (MRI) system. The apparatus comprises: at least one first B₀ magnet configured to produce a first magnetic field to contribute to the B₀ magnetic field for the MRI system, the at least one first B₀ magnet comprising ferromagnetic rings including a first ferromagnetic ring and a second ferromagnetic ring, the first ferromagnetic ring having a first magnetization and the second ferromagnetic ring having a second magnetization. The first magnetization and the second magnetization have first radial and axial components and second radial and axial components, respectively; and the first radial and axial components are different than the second radial and axial components.

### BRIEF DESCRIPTION OF DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIG. 1 illustrates exemplary components of a magnetic resonance imaging (MRI) system, in accordance with some embodiments of the technology described herein;

FIG. 2 illustrates a schematic of a cross section of an ideal Halbach cylinder;

FIG. 3 illustrates an example of a cylindrical coordinate system;

FIG. 4 illustrates an example of a cylindrical magnet assembly for providing a B₀ magnetic field for an MRI system, in accordance with some embodiments of the technology described herein;

FIG. 5A illustrates an example of a cylindrical magnet assembly including regions of differing magnetization for providing a B₀ magnetic field for an MRI system, in accordance with some embodiments of the technology described herein;

FIG. 5B illustrates an example of another cylindrical magnet assembly including regions of differing magnetization for providing a B₀ magnetic field for an MRI system, in accordance with some embodiments of the technology described herein;

FIG. 6 illustrates an example of a bi-planar magnet assembly for providing a B₀ magnetic field for an MRI system, in accordance with some embodiments of the technology described herein;

FIG. 7 illustrates an example of magnetic orientations within the bi-planar magnet assembly of FIG. 6, in accordance with some embodiments of the technology described herein;

FIG. 8 illustrates an example of a C-shaped frame configured to support the bi-planar magnet assembly of FIG. 6, in accordance with some embodiments of the technology described herein;

FIGS. 9-11 illustrate examples of frames configured to support the bi-planar magnet assembly of FIG. 6, in accordance with some embodiments of the technology described herein;

FIG. 12 illustrates an MRI system including the magnet assembly of FIG. 8, in accordance with some embodiments of the technology described herein;

FIG. 13 illustrates the use of the MRI system of FIG. 10 to image a patient's head, in accordance with some embodiments of the technology described herein; and

FIG. 14 depicts, schematically, an illustrative computing device on which aspects of the technology described herein may be implemented.

### DETAILED DESCRIPTION

Conventional magnetic resonance imaging (MRI) systems are overwhelmingly high-field systems, particularly for medical or clinical MRI applications. The general trend in medical imaging has been to produce MRI scanners with increasingly greater field strengths, with the vast majority of clinical MRI scanners operating at 1.5 T or 3 T, with higher field strengths of 7 T and 9 T used in research settings. As used herein, “high-field” refers generally to MRI systems presently in use in a clinical setting and, more particularly, to MRI systems operating with a main magnetic field (i.e., a B₀ field) at or above 1.5 T, though clinical systems operating between 0.5 T and 1.5 T are often also characterized as “high-field.” By contrast, “low-field” refers generally to MRI systems operating with a B₀ field of less than or equal to approximately 0.2 T, though systems having a B₀ field of between 0.2 T and approximately 0.3 T have sometimes been characterized as low-field as a consequence of increased field strengths at the high end of the high-field regime.

Some conventional low-field MRI systems may produce the main magnetic B₀ field using one or more permanent magnets. A permanent magnet may be any object or material that maintains its own persistent magnetic field once magnetized. Materials that can be magnetized to produce a permanent magnet are referred to herein as ferromagnetic and include, as non-limiting examples, iron, nickel, cobalt, neodymium alloys (e.g., NdFeB), samarium cobalt (SmCo) alloys, alnico (AlNiCo) alloys, strontium ferrite, barium ferrite, etc.

Permanent magnets are conventionally manufactured using powder metallurgy methods. In this process, a suitable permanent magnet material is pulverized into a fine powder, compacted, and heated to sinter the powder into a solid piece. Conventionally, such sintered magnets are compacted in a hydraulic or mechanical press, which can limit the shape of the resulting sintered magnets to simple cross-sections that can be pushed out of the die cavity.

Recently, demonstrations of sintered magnets formed using a swaging manufacturing process have been made. As used herein, a swaging manufacturing process refers to any process for manufacturing a permanent magnet that involves forming the permanent magnet by swaging its constituent material (e.g., metallic powder) while applying a magnetic field to the constituent material. In some embodiments, the swaged and magnetized materials may then be sintered to form a solid component. In other embodiments, the constituent material may include a bonding agent so that a separate sintering step is not needed. In such embodiments, the bonding agent may be used to programmably reduce a net magnetization of a region of the resulting solid component. After the swaged material is cooled, it may be further magnetized (e.g., using an electromagnet, an array of permanent magnets) to have a desired magnetization direction or directions. Examples of swaging manufacturing processes are described in U.S. Patent Application Publication No. 2019/0122818, filed Sep. 28, 2018, and titled “Method of Manufacturing Permanent Magnets,” and U.S. Patent Application Publication No. 2018/0226190, filed Mar. 30, 2018, and titled “Single-Step Manufacturing of Flux-Directed Permanent Magnet Assemblies,” each of which is incorporated by reference herein in its entirety. Such swaging manufacturing processes enable the production of long lengths of magnet block in a rapid and efficient manner.

Additionally, such swaging manufacturing processes enable the production of hollow magnetic structures integrally formed as a single component. In particular, these hollow magnetic structures may be magnetized during the manufacturing process to form continuous flux Halbach cylinders. These continuous flux Halbach cylinders have been implemented in electric propulsion motor applications, as described in the '818 and '190 patent application publications identified in the foregoing paragraph.

Although these swaging manufacturing processes have been implemented for producing components for electric propulsion motors, the inventors have recognized that these swaging manufacturing processes may be adapted for manufacturing permanent magnets for MRI applications. For example, MRI applications may rely on substantially homogenous B₀ magnetic fields to produce high-resolution and/or otherwise clinically useful magnetic resonance (MR) images. However, the finite length of such hollow magnetic structures may create non-homogenous magnetic fields. The inventors have recognized that such non-homogeneity may be compensated for, by way of example, by splitting the permanent magnet into multiple rings of tailored length, separated by gaps and/or permanent magnets of opposing polarity. The inventors have recognized that such rings could be manufactured using a swaging manufacturing process.

Accordingly, the inventors have developed methods and systems for providing a B₀ magnetic fields within an MRI system using one or more permanent magnets and/or permanent magnetic assemblies formed using a swaging manufacturing process. In some embodiments, the apparatus for providing a B₀ magnetic field for an MRI system may include a cylindrical shell forming a bore extending along a common longitudinal direction. The cylindrical shell may include a first plurality of ferromagnetic rings including a first ferromagnetic ring with an angularly varying magnetization orientation. The cylindrical shell may also include a second plurality of rings. In some embodiments, the first ferromagnetic ring may be manufactured by swaging. In some embodiments, at least one (e.g., one, some or all) of the first plurality of ferromagnetic rings may be manufacturing by swaging.

In some embodiments, the second plurality of rings also includes ferromagnetic rings. The ferromagnetic rings may include a second ferromagnetic ring having an angularly varying magnetization orientation. In some embodiments, the angularly varying magnetization of the first ferromagnetic ring and that of the second ferromagnetic rings angularly vary in opposing directions. In other embodiments, the second plurality of rings includes non-ferromagnetic rings (e.g., spacers made from non-ferrous material such as plastic, fiberglass, etc.).

In some embodiments, the first plurality of ferromagnetic rings may be interspersed with the second plurality of rings (e.g., the first plurality of ferromagnetic rings may be alternatingly arranged between rings of the second plurality of rings).

In some embodiments, each of the first plurality of ferromagnetic rings and each of the second plurality of rings may have the same diameter to provide a bore having a constant diameter along the common longitudinal direction. In other embodiments, at least two of the plurality of ferromagnetic rings may have different inner diameters (e.g., to provide a bore with a changing diameter along its length). For example, rings of the first plurality of ferromagnetic rings and second plurality of rings may have differing diameters along the length of the bore so that bore's diameter is larger at one end of the bore than at another end of the bore.

In some embodiments, the B₀ magnetic field has a field strength that is greater than 0.02 T and less than 0.2 T. In some embodiments, the B₀ magnetic field has a field strength that is greater than 0.05 T and less than 0.1 T. In some embodiments, the B₀ magnetic field has a field strength that is greater than 0.06 T and less than 0.07 T.

In some embodiments, the B₀ magnetic field has a homogeneity less than or equal to substantially 1000 ppm within an imaging region disposed within the bore. In some embodiments, the B₀ magnetic field has a homogeneity less than or equal to substantially 500 ppm, less than or equal to substantially 250 ppm, less than or equal to substantially 100 ppm, or less than or equal to substantially 50 ppm within an imaging region disposed within the bore. In other embodiments, the B₀ magnetic field has a homogeneity of substantially 10 ppm within an imaging region disposed within the bore. Alternatively, in some embodiments, the B₀ magnetic field has a homogeneity in a range from 100 ppm to 1000 ppm, from 500 ppm to 1000 ppm, 100 ppm to 500 ppm, from 5 ppm to 100 ppm, or any other suitable range within these ranges within an imaging region disposed within the bore

In some embodiments, the apparatus includes less than 400 kg of permanent magnet material. In some embodiments, the apparatus includes less than 300 kg of permanent magnet material, less than 200 kg of permanent magnet material, less than 100 kg of permanent magnetic material, or less than 40 kg of permanent magnet material. In other embodiments, the apparatus includes between 300 kg to 500 kg of permanent magnet material, between 100 kg to 400 kg of permanent magnet material, between 10 kg and 100 kg of permanent magnet material, or any other suitable range within these ranges.

The inventors have also developed an MRI system including an apparatus for providing a B₀ magnet field using one or more permanent magnets formed using a swaging manufacturing process. In some embodiments, the apparatus may include a cylindrical shell forming a bore extending along a common longitudinal direction. The cylindrical shell may include a first plurality of ferromagnetic rings including a first ferromagnetic ring with an angularly varying magnetization orientation. The cylindrical shell may also include a second plurality of rings. In some embodiments, the MRI system may also include gradient coils configured to, when operated, generate magnetic fields to provide spatial encoding of emitted magnetic resonance signals, at least one radio frequency (RF) transmit coil, and a power system configured to provide power to the gradient coils and the at least one RF transmit coil.

The inventors have further developed methods of manufacturing an apparatus for providing a B₀ magnet field within an MRI system using one or more permanent magnets formed using a swaging manufacturing process. In some embodiments, the method may include manufacturing a ferromagnetic cylinder at least in part by placing a magnetic metal alloy powder in an annular volume between an outer cylindrical tube and an inner cylindrical tube and applying a magnetic field to the magnetic metal alloy powder while compressing the magnetic metal alloy powder. The method may include bonding (e.g., sintering, using a binding compound, etc.) the magnetic metal alloy powder to form at least a part of the ferromagnetic cylinder and magnetizing the ferromagnetic cylinder to have an angularly varying magnetization orientation.

In some embodiments, the method may include partitioning the ferromagnetic cylinder into a first plurality of ferromagnetic rings and assembling, from the first plurality of ferromagnetic rings and a second plurality of rings, a cylindrical shell forming a bore extending along a common longitudinal direction. In some embodiments, the second plurality of ferromagnetic rings may include one or more ferromagnetic rings with an angularly varying magnetization orientation. In some such embodiments, the first plurality of ferromagnetic rings may include a first ferromagnetic ring with an angularly varying magnetization orientation, and the second plurality of rings may include a second ferromagnetic ring with an angularly varying magnetization orientation. The magnetization orientations of the first and second ferromagnetic rings may angularly vary in opposing directions. In other embodiments, the second plurality of rings comprises one or more non-ferromagnetic rings.

In some embodiments, assembling of the ferromagnetic cylinder may include using the second plurality of rings as spacers among rings in the first plurality of ferromagnetic rings so that the first plurality of ferromagnetic rings are interspersed with the second plurality of rings (e.g., so that rings of the first plurality of ferromagnetic rings alternate with rings of the second plurality of rings along the length of the ferromagnetic cylinder).

In other embodiments, the method of manufacturing the apparatus may include manufacturing a ferromagnetic cylindrical shell at least in part by placing magnetic metal alloy powder and non-ferromagnetic powder in an annular volume between an outer cylindrical tube and an inner cylindrical tube. The method may include applying a magnetic field to the magnetic metal alloy powder while compressing the magnetic metal alloy powder and bonding the magnetic metal alloy powder and the non-ferromagnetic powder to form the ferromagnetic cylindrical shell. The method may further include magnetizing the ferromagnetic cylindrical shell to have an angularly varying magnetization orientation. In such embodiments, placing the non-magnetic powder between the two cylindrical tubes may include interspersing the non-magnetic powder with the magnetic metal alloy powder. The method may further include removing the two cylindrical tubes from the ferromagnetic cylindrical shell.

In other embodiments, the method of manufacturing the apparatus may include manufacturing a ferromagnetic cylindrical shell at least in part by placing magnetic metal alloy powder in an annular volume between an outer cylindrical tube and an inner cylindrical tube and applying a magnetic field to the magnetic metal alloy powder while compressing the magnetic metal alloy powder. The method may further include bonding the magnetic metal alloy powder and the non-ferromagnetic powder to form the ferromagnetic cylindrical shell and selectively magnetizing first ring regions of the ferromagnetic cylinder to have a first angularly varying magnetization orientation. Second ring regions of the ferromagnetic cylindrical shell may be selectively magnetized to have a second angularly varying magnetization orientation. The second angularly varying magnetization orientation may vary in a direction opposing that of the first angularly varying magnetization orientation. In some embodiments, the first ring regions may be interspersed with the second ring regions. In some embodiments, the method may further include removing the two cylindrical tubes from the cylindrical shell.

Alternatively, the inventors have recognized that permanent magnets formed using a swaging manufacturing process may be used in place of conventional permanent magnet blocks to form planar permanent magnet assemblies. The inventors have accordingly developed an apparatus for providing a B₀ magnetic field for a magnetic resonance imaging (MRI) system, the apparatus may include at least one first B₀ magnet configured to produce a first magnetic field to contribute to the B₀ magnetic field for the MRI system.

In some embodiments, the at least one first B₀ magnet may include ferromagnetic rings, the ferromagnetic rings including a first ferromagnetic ring and a second ferromagnetic ring. The first ferromagnetic ring may have a first magnetization, and the second ferromagnetic ring may have a second magnetization, and the first magnetization and the second magnetization may have first radial and axial components and second radial and axial components, respectively. In some embodiments, the first radial and axial components may be different than the second radial and axial components.

In some embodiments, the first ferromagnetic ring may be integrally formed as a single monolithic component. For example, the first ferromagnetic ring may be manufactured at least in part by swaging.

In some embodiments, the ferromagnetic rings may have different heights. In some embodiments, the ferromagnetic rings may be concentric rings having different diameters.

Following below are more detailed descriptions of various concepts related to, and embodiments of swaged magnet assemblies. It should be appreciated that various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

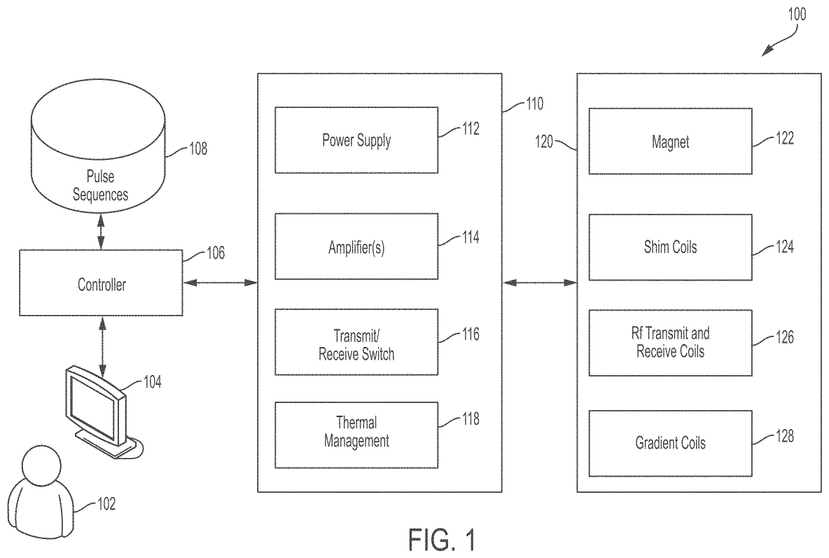

FIG. 1 is a block diagram of components of a MRI system 100. In the illustrative example of FIG. 1, MRI system 100 comprises computing device 104, controller 106, pulse sequences store 108, power management system 110, and magnetics components 120. It should be appreciated that system 100 is illustrative and that an MRI system may have one or more other components of any suitable type in addition to or instead of the components illustrated in FIG. 1. However, an MRI system will generally include these high level components, though the implementation of these components for a particular MRI system may differ.

As illustrated in FIG. 1, magnetics components 120 comprise B₀ magnet 122, shim coils 124, RF transmit and receive coils 126, and gradient coils 128. Magnet 122 may be used to generate the main magnetic field B₀. Magnet 122 may be any suitable type or combination of magnetics components that can generate a desired main magnetic B₀ field. In some embodiments, magnet 122 may be a permanent magnet, an electromagnet, a superconducting magnet, or a hybrid magnet comprising one or more permanent magnets and one or more electromagnets and/or one or more superconducting magnets. In some embodiments, magnet 122 may be a bi-planar permanent magnet and, in some embodiments, may include multiple sets of concentric permanent magnet rings. In some embodiments, magnet 122 may include one or more permanent magnets manufactured using swaging techniques, as described herein.

Gradient coils 128 may be arranged to provide gradient fields and, for example, may be arranged to generate gradients in the B₀ field in three substantially orthogonal directions (X, Y, and Z). Gradient coils 128 may be configured to encode emitted MR signals by systematically varying the B₀ field (the B₀ field generated by magnet 122 and/or shim coils 124) to encode the spatial location of received MR signals as a function of frequency or phase. For example, gradient coils 128 may be configured to vary frequency or phase as a linear function of spatial location along a particular direction, although more complex spatial encoding profiles may also be provided by using nonlinear gradient coils.

MRI is performed by exciting and detecting emitted MR signals using transmit and receive coils, respectively (often referred to as radio frequency (RF) coils). Transmit/receive coils may include separate coils for transmitting and receiving, multiple coils for transmitting and/or receiving, or the same coils for transmitting and receiving. Thus, a transmit/receive component may include one or more coils for transmitting, one or more coils for receiving and/or one or more coils for transmitting and receiving. Transmit/receive coils are also often referred to as Tx/Rx or Tx/Rx coils to generically refer to the various configurations for the transmit and receive magnetics component of an MRI system. These terms are used interchangeably herein. In FIG. 1, RF transmit and receive coils 126 comprise one or more transmit coils that may be used to generate RF pulses to induce an oscillating magnetic field B₁. The transmit coil(s) may be configured to generate any suitable types of RF pulses.

Power management system 110 includes electronics to provide operating power to one or more components of the low-field MRI system 100. For example, power management system 110 may include one or more power supplies, gradient power components, transmit coil components, and/or any other suitable power electronics needed to provide suitable operating power to energize and operate components of MRI system 100. As illustrated in FIG. 1, power management system 110 comprises power supply 112, power component(s) 114, transmit/receive switch 116, and thermal management components 118 (e.g., cryogenic cooling equipment for superconducting magnets). Power supply 112 includes electronics to provide operating power to magnetic components 120 of the MRI system 100. For example, power supply 112 may include electronics to provide operating power to one or more B₀ coils (e.g., B₀ magnet 122) to produce the main magnetic field for the low-field MRI system. Transmit/receive switch 116 may be used to select whether RF transmit coils or RF receive coils are being operated.

Power component(s) 114 may include one or more RF receive (Rx) pre-amplifiers that amplify MR signals detected by one or more RF receive coils (e.g., coils 126), one or more RF transmit (Tx) power components configured to provide power to one or more RF transmit coils (e.g., coils 126), one or more gradient power components configured to provide power to one or more gradient coils (e.g., gradient coils 128), and one or more shim power components configured to provide power to one or more shim coils (e.g., shim coils 124).

As illustrated in FIG. 1, MRI system 100 includes controller 106 (also referred to as a console) having control electronics to send instructions to and receive information from power management system 110. Controller 106 may be configured to implement one or more pulse sequences, which are used to determine the instructions sent to power management system 110 to operate the magnetic components 120 in a desired sequence (e.g., parameters for operating the RF transmit and receive coils 126, parameters for operating gradient coils 128, etc.). As illustrated in FIG. 1, controller 106 also interacts with computing device 104 programmed to process received MR data. For example, computing device 104 may process received MR data to generate one or more MR images using any suitable image reconstruction process(es). Controller 106 may provide information about one or more pulse sequences to computing device 104 for the processing of data by the computing device. For example, controller 106 may provide information about one or more pulse sequences to computing device 104 and the computing device may perform an image reconstruction process based, at least in part, on the provided information.

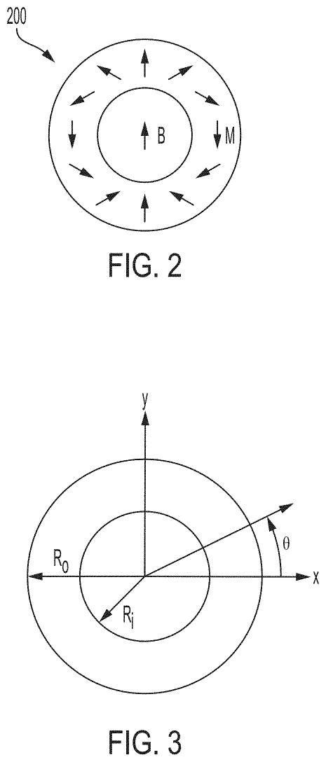

FIG. 2 illustrates a schematic of a cross section of an ideal Halbach cylinder 200. In such an ideal Halbach cylinder 200, the permanent magnet material forms a cylindrical structure in which the magnetization, M, rotates twice as fast as the position rotates around the cylinder (e.g., the magnetization makes two complete rotations around the circumference of the cylinder). In the ideal two-dimensional case, the cylinder is infinitely long and the magnetization rotates continuously around the cylinder, creating a uniform magnetic field, B, within its cavity and generating zero field outside the cylinder. Although these characteristics are highly desirable, they are difficult to achieve in practice. Indeed, in practice, the cylinder has finite length and a continuous variation of magnetization orientation around the cylinder is difficult to achieve in manufacturing. Rather, conventional Halbach arrays are manufactured by: (1) discretizing the cylinder in the azimuthal direction and/or along the axis of the cylinder into multiple blocks, each of which is easier to manufacture; and (2) assembling the Halbach array out of the multiple blocks. However, the inventors have recognized that a swaging manufacturing process may be used to form cylindrical shells with continuous or nearly-continuous variation of magnetization orientation which may be used for MRI applications.

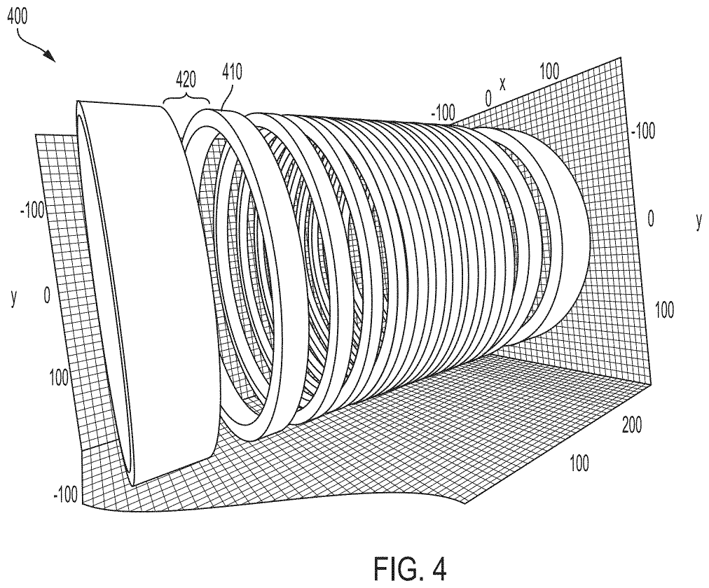

FIG. 3 illustrates an example of a cylindrical shell 300 within a cylindrical coordinate system. The cylindrical shell 300 has a cross section which is parallel to the x-y plane, while the cylindrical shell extends along a common longitudinal direction parallel to the z-direction (e.g., out of the page). The angular position within the cylindrical shell 300 is defined by an angle, θ, relative to the x-axis. The cylindrical shell 300 also has a thickness defined by a difference between an inner radius, Rᵢ, and an outer radius, Rₒ.

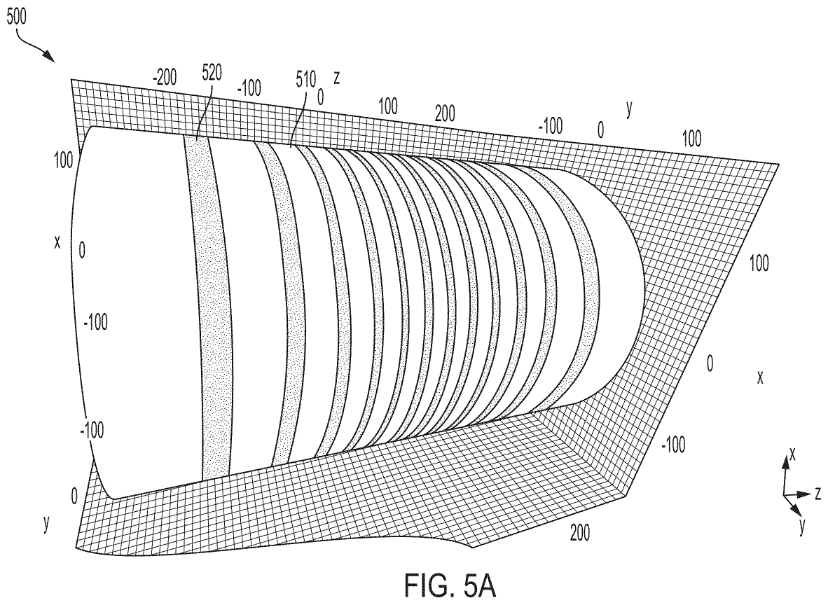

FIG. 4 illustrates an example of a cylindrical magnet assembly 400 for providing a B₀ magnetic field for an MRI system, in accordance with some embodiments of the technology described herein. The cylindrical magnet assembly 400 includes ferromagnetic rings 410 and non-ferromagnetic regions 420. The ferromagnetic rings 410 and non-ferromagnetic regions 420 may be tailored in length and spacing to provide a substantially homogenous magnetic field in a central region (e.g., imaging region) of the cylindrical magnet assembly 400. The length, spacing, and arrangement of ferromagnetic rings 410 and non-ferromagnetic regions 420 may be determined using computational optimization methods, as described herein.

In some embodiments, the ferromagnetic rings 410 may be formed of any suitable permanent magnet material, examples of which are described herein. The ferromagnetic rings 410 may be formed by a swaging manufacturing process. For example, the ferromagnetic rings 410 may be individually manufactured by a swaging manufacturing process. Alternatively, the ferromagnetic rings 410 may be cut from a larger, integrally-formed piece of cylindrical stock formed by a swaging manufacturing process.

The swaging manufacturing process may use metallic tubes to form the ferromagnetic cylindrical shells, in some embodiments. Such metallic tubes, if left on the ferromagnetic rings 410 after manufacturing, may provide a conduction path for eddy currents caused by gradient field pulses within the MRI system. Accordingly, in some embodiments, such tubing is removed or partially removed (e.g., by abrasion, etching, or other techniques) prior to assembly of magnet assembly 400 to reduce eddy currents along the surfaces of the ferromagnetic rings 410. Alternatively, using non-metallic tubes (e.g., plastic) or poorly conducting metal tubes (e.g., tungsten) may eliminate or reduce the effect of eddy currents in a final MRI system including magnet assembly 400.

In some embodiments, the ferromagnetic rings 410 may have a continuously rotating magnetic orientation. The magnetic orientation may vary with angular position, θ, within the ferromagnetic rings 410. For example, the ferromagnetic rings may have a continuously rotating magnetic orientation similar to that of the ideal Halbach cylinder 200, as described in connection with FIG. 2.

In some embodiments, the non-ferromagnetic regions 420 may be gaps (e.g., air gaps) between the ferromagnetic rings 410. Alternatively, the non-ferromagnetic regions 420 may include spacer rings formed of one or more non-magnetic materials (e.g., plastic, fiberglass). The spacer rings may be formed of transparent material to reduce claustrophobia of a patient within the MRI system during an MRI procedure.

In some embodiments, the magnet assembly 400 may be assembled by interspersing individual ferromagnetic rings 410 with individual non-ferromagnetic regions 420. In other embodiments, the magnet assembly 400 may formed integrally as a single unit using a swaging manufacturing process. The magnet assembly 400 may be formed integrally as a single unit by interspersing regions of ferromagnetic powder alloy with regions of non-ferromagnetic powder alloy during the swaging manufacturing process.

In some embodiments, the magnet assembly 400 may be asymmetrical along the common longitudinal direction of the bore. For example, the inner diameter of the magnet assembly 400 may be larger at one end of the magnet assembly 400 than at the other end of the magnet assembly 400. Such asymmetrical embodiments may enable a shorter distance from an entrance of the cylinder to the imaging region of the MRI system. This shorter distance may increase accessibility of the MRI system for patients and/or users.

The magnet assembly 400 may have a minimum length to obtain substantial field homogeneity within a suitably-sized imaging region (e.g., approximately within a volume having a diameter ranging from 15 cm to 30 cm). The dimensions of magnet assembly 400 may be scaled by the same ratio in all dimensions and preserve its magnetic properties (e.g., field strength, homogeneity, etc.). Herein, the dimensions of magnet assembly 400 will be expressed in terms of one reference dimension taken as unity. A convenient reference dimension is the inner radius of magnet assembly 400. If the inner radius is set to 1, and the desired imaging region has a radius of 0.7 (e.g., 70% of the inner diameter of the magnet assembly 400 may be usable for imaging), then the minimum length of magnet assembly 400 may be approximately 3.5 with an outer radius of 1.1, which minimizes volume. With these dimensions, the magnet assembly 400 of FIG. 4 may offer a B₀ magnetic field of 60 mT with approximately 10 ppm homogeneity over the imaging region volume.

However, such a structure may be limiting in terms of access. The diameter-to-length ratio (0.57) may be quite small, and a patient's head may not be able to be positioned in the imaging region if the patient's shoulders cannot fit within the bore. Such a structure may need to be increased to a typical MRI bore diameter to allow access for most patients. Accordingly, in some embodiments, for a magnet assembly 400 with an inner radius of 350 mm, the magnet assembly 400 may include approximately 380 kg of permanent magnet material.

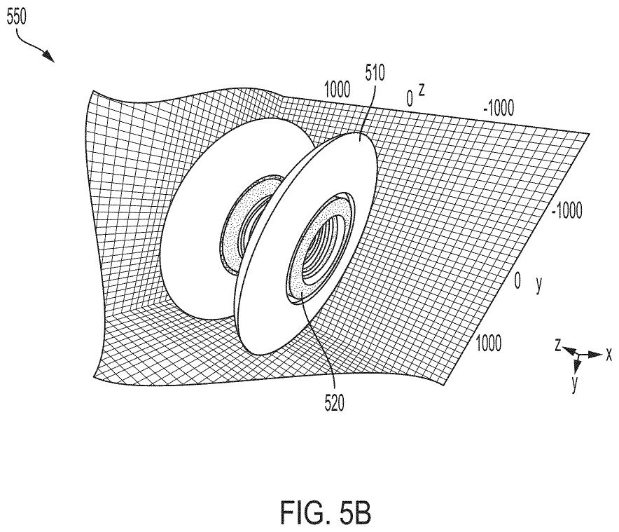

FIG. 5A illustrates an example of a cylindrical magnet assembly 500, including regions of differing magnetization, for providing a B₀ magnetic field for an MRI system, in accordance with some embodiments of the technology described herein. Cylindrical magnet assembly 500 includes first ferromagnetic rings 510 and second ferromagnetic rings 520. First ferromagnetic rings 510 and second ferromagnetic rings 520 may be formed of any suitable permanent magnet material, as described herein. First ferromagnetic rings 510 and second ferromagnetic rings 520 may be formed of a same permanent magnet material, in some embodiments, while in other embodiments first ferromagnetic rings 510 and second ferromagnetic rings 520 may be formed of different permanent magnet materials (e.g., to provide differently-sized contributions to the B₀ magnetic field).

In some embodiments, the first ferromagnetic rings 510 and second ferromagnetic rings 520 may have angularly-varying magnetizations. For example, the first ferromagnetic rings 510 and the second ferromagnetic rings 520 may have a continuously-rotating magnetization as in the example of Halbach cylinder 200. In some embodiments, the first ferromagnetic rings 510 and second ferromagnetic rings 520 may have magnetizations with opposing polarities. Such embodiments may enable a shorter magnet assembly (e.g., having a shorter length along the common longitudinal direction) than a cylindrical magnet assembly including ferromagnetic rings and non-ferromagnetic regions (e.g., as described in relation to magnet assembly 400).

In some embodiments, first ferromagnetic rings 510 and second ferromagnetic rings 520 may be formed using a swaging manufacturing process. The first ferromagnetic rings 510 and second ferromagnetic rings 520 may be formed individually prior to assembling magnet assembly 500. In such embodiments, stock ferromagnetic cylindrical shells with a desired inner and outer radii and a desired magnetization pattern may be formed using a swaging manufacturing process. The stock ferromagnetic cylindrical shells may be sliced into ferromagnetic rings of desired lengths, and the ferromagnetic rings may be assembled to form magnet assembly 500. The ferromagnetic rings may be rotated relative to each other to appropriately orient the magnetization patterns (e.g., to orient the polarity of first ferromagnetic rings 510 in a first manner and to orient the polarity of second ferromagnetic rings 520 in a second, opposing manner).

In other embodiments, magnet assembly 500 may be integrally formed as a single piece using a swaging manufacturing process. For example, in such embodiments, a single ferromagnetic cylindrical shell may be manufactured. During a process of magnetizing the ferromagnetic cylindrical shell, the magnetic alignment fixture (e.g., electromagnetic coils, permanent magnet fixtures) may be rotated in order to change the polarity of the ferromagnetic cylindrical shell as a function of position along the common longitudinal direction (e.g., along the z-axis) of the ferromagnetic cylindrical shell. Alternatively, the polarity of the magnetic alignment fixture may be reversed by changing the direction of current flow (e.g., for electromagnetic alignment fixtures).

In some embodiments, a magnet assembly may also be discretized along the radial direction in addition to along the common longitudinal direction in order to increase the available degrees of freedom. An example of such a magnet assembly is magnet assembly 550 shown in FIG. 5B, in accordance with some embodiments of the technology described herein. Such discretization along the radial direction may enable a shorter length along the common longitudinal direction.

Magnet assembly 550 includes first ferromagnetic rings 510 and second ferromagnetic rings 520, the first ferromagnetic rings 510 having opposing polarities as the second ferromagnetic rings 520. In such embodiments, the reduction of length along the common longitudinal direction may come at the cost of increased permanent magnet material weight. For example, for an inner radius of 350 mm and length of 700 mm (e.g., a 1:1 aspect ratio), the magnet assembly 550 may include approximately 4000 kg of permanent magnet material. For an inner radius of 150 mm, the magnet assembly 550 may include approximately 300 kg of permanent magnet material.

In some embodiments, computational optimization methods may be used to determine the layouts of the magnet assemblies (e.g., magnet assemblies 400, 500, and/or 550). Such computational optimization methods may be performed using any suitable computing environment executing suitable optimization software.

In some embodiments, linear programming may be used to generate the magnet assembly layouts including ferromagnetic rings and non-ferromagnetic regions (e.g., magnet assembly 400). In such embodiments, a cylindrical shell may be defined in which magnetic material can be present. The geometric structure of the magnetic assembly may be constrained to have axial symmetry. The magnetization within the cylindrical shell may be constrained to be continuous and/or Halbach around the axis of symmetry, called O_{z} herein, so that the magnetic field in the bore of the cylindrical shell is along Oₓ. The region of space where magnetized material can be positioned is located between points −b₀ and +b₀ along the common longitudinal axis and between p₁ and p₂ radially. This space may be discretized along the axis O_{z} into tubular slices. In some embodiments, the structure may be constrained to be symmetric with respect to the plane xOy. It can be shown that the scalar potential Φ* from which the field component Bₓ in the cavity derives is of the form:

$${Φ}^{*} ( r , θφ ) = {\sum }_{n = 1}^{∞} {r}^{n} X {Φ}_{n}^{1} cos φ {P}_{n}^{1} ( cos θ )$$

The main component of the field Bₓ may then be given by:

$${B}_{x} = {Z}_{0} + {\sum }_{n = 1}^{∞} {r}^{n} ( {Z}_{n} {P}_{n} ( cos θ ) + {X}_{n}^{2} {P}_{n}^{2} ( cos θ ) cos ( 2 φ ) \\ where : \\ {Z}_{n} = - {μ}_{0} \frac{( n + 1 ) ( n + 2 )}{2} X {Φ}_{n + 1}^{1} \\ {X}_{n}^{2} = \frac{{μ}_{0}}{2} X {Φ}_{n + 1}^{1}$$

Obtaining a homogeneous field in the imaging region may require canceling as many XΦₙ¹ terms as necessary, with n>1. The symmetry in xOy eliminates every even term of the potential (e.g., every odd term of the field). The optimization may be performed by building a matrix of effect of each tube slice on each term to be considered which we call W, with elements wₙⁱ where n is the order of the term and i is the index of the slice. The goal is to minimize the amount of material used within the allowed space to achieve a given field strength and homogeneity. This can be treated with a linear programming approach, where the linear program problem may be described by the objective function:

$$\frac{min imize}{X} {\sum }_{i} {x}_{i} {v}_{i}$$

subject to the following linear constraints:

$${b}_{0} - Δ b \leq {\sum }_{i} {w}_{1}^{i} {x}_{i} \leq {b}_{0} + Δ b \\ - δ b \leq {\sum }_{i} {w}_{n}^{i} {x}_{i} \leq + δ b for all 2 \leq n \leq N$$

$$0 \leq {x}_{i} \leq 1$$

where X is the vector of material density in a slice, V is the vector of volume for each slice, b₀ is the desired field, Δb is a tolerance on the desired field strength, and δb is a tolerance of deviation for each term, which translates to a maximum field variation at a given radius. N is the maximum order to be considered for the terms.

In other embodiments including two ferromagnetic rings of opposing polarities, the linear program may be augmented by variables and constraints to describe the “negative” polarization. A vector D of positive variables may be introduced. The vector D may have components dᵢ, and the vector D may be used for the objective function in the same manner as previously and may be used to constrain the free variable X, such that the linear optimization problem may be described by the objective function:

$$\frac{min imize}{D , X} {\sum }_{i} {d}_{i} {v}_{i}$$

subject to the following linear constraints:

$${b}_{0} - Δ b \leq {\sum }_{i} {w}_{1}^{i} {x}_{i} \leq {b}_{0} + Δ b \\ - δ b \leq {\sum }_{i} {w}_{n}^{i} {x}_{i} \leq + δ b for all 2 \leq n \leq N \\ - {d}_{i} \leq {x}_{i} \leq + {d}_{i} ∀ i$$

$$0 \leq {d}_{i} \leq 1$$

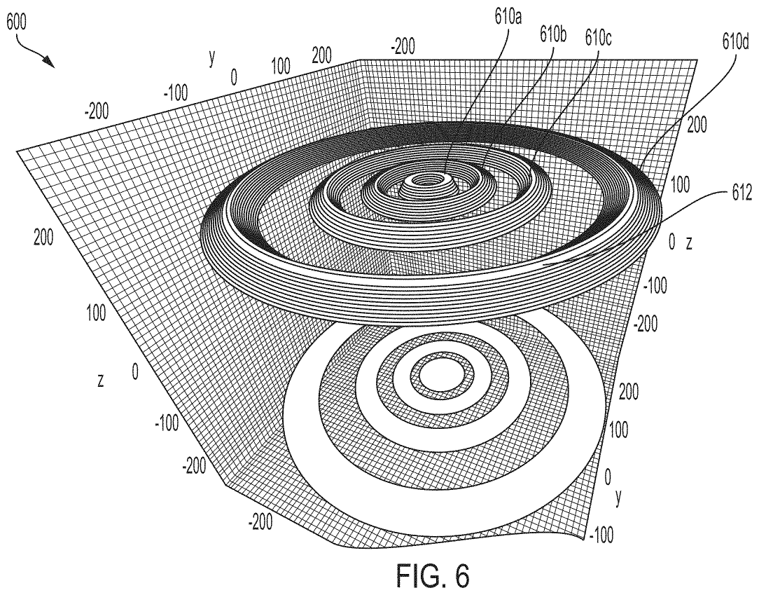

The inventors have recognized that a swaging manufacturing process may also be used to make magnetic assemblies for MRI systems that may be supported by a ferromagnetic frame, sometimes termed a “yoke.” FIG. 6 illustrates an example of a magnet assembly 600 for providing a B₀ magnetic field for an MRI system, in accordance with some embodiments of the technology described herein. The magnet assembly 600 may include one or more sets of concentric ferromagnetic rings 610a-d. The magnet assembly 600 may be bi-planar in configuration, with two sets of opposing ferromagnetic rings 610a-d, as shown in FIG. 6. Alternatively, the magnet assembly 600 may only have one set of ferromagnetic rings 610a-d. In some embodiments, one or more of ferromagnetic rings 610a-d may have different heights. In other embodiments, one or more of ferromagnetic rings 610a-d may have substantially the same heights.

In some embodiments, the magnet assembly 600 may be configured to provide a B₀ magnetic field in a range from 0.05 T to 0.2 T. Additionally or alternatively, magnet assembly 600 may be configured to provide a B₀ magnetic field in a range from 0.05 T to 0.1 T. In other embodiments, magnet assembly 600 may be configured to provide a B₀ magnetic field of 64 mT.

In some embodiments, one or more of the ferromagnetic rings 610a-d may be formed of ferromagnetic sub-rings 612. In some embodiments, ferromagnetic sub-rings 612 may be formed of any suitable permanent magnetic materials, as described herein. The ferromagnetic sub-rings 612 may be manufactured using a swaging manufacturing process, such as a process as described in the '818 and '190 patent applications described previously herein. For example, stock ferromagnetic cylindrical shells having desired inner and outer diameters may be formed using a swaging manufacturing process, and the ferromagnetic cylindrical shells may be partitioned into individual ferromagnetic sub-rings 612. The ferromagnetic sub-rings 612 may be assembled to form one or more of ferromagnetic rings 610a-d. Alternatively, in some embodiments, one or more of the ferromagnetic rings 610a-d may be monolithically formed as a solid ring having a uniform magnetization rather than comprising an assembly of ferromagnetic sub-rings 612.

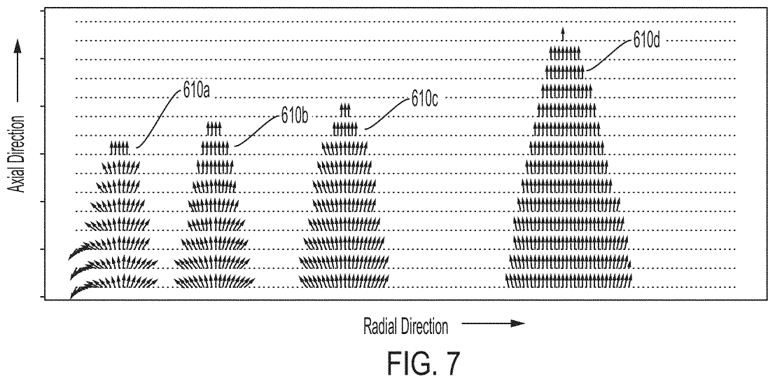

In some embodiments, each ferromagnetic sub-ring 612 may have a uniform magnetization through its volume. The magnetization of each ferromagnetic sub-ring 612 may have radial and/or axial components, may have only a radial component, or may have only an axial component. In some embodiments, ferromagnetic sub-rings 612 may have different magnetizations with different radial and axial components from each other. In such embodiments the ferromagnetic rings 610a-d may have varying magnetization orientations within their assemblies, as shown in the example of FIG. 7.

FIG. 7 depicts an example of magnetic orientations of ferromagnetic sub-rings 612 within each ferromagnetic ring 610a-d, in accordance with some embodiments of the technology described herein. The cross-section of FIG. 7 is shown along the radial and axial directions, as the magnetization is substantially the same at each angular position within magnet assembly 600. The magnetic orientation of each ferromagnetic sub-ring 612 is represented in FIG. 7 by an arrow and may have axial and/or radial components. The magnetic orientations may be determined based on a desired B₀ magnetic field strength and/or field homogeneity.

In some embodiments, ferromagnetic rings 610a-d may have triangle-like radial cross-sections, as may be seen in FIG. 7. Such triangle-like cross-sections are difficult to produce using traditional sintered magnet blocks, but may reduce the amount of permanent magnetic material needed to produce a same B₀ magnetic field (e.g., such a triangle-like cross-section may increase B₀ magnetic field efficiency). For example, magnet assembly 600 may include less than 40 kg of permanent magnetic material to produce a same B₀ magnetic field as a conventional magnet assembly with a rectangular cross-section which includes 60 kg of permanent magnetic material. Alternatively, in some embodiments, one or more of ferromagnetic rings 610a-d may have a rectangular cross-section (not shown) in a plane parallel to the axial and radial directions.

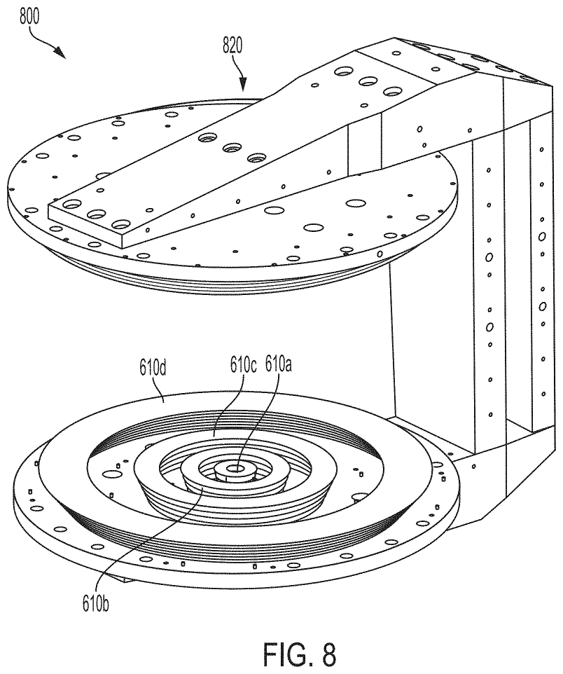

When integrated into an MRI system, the magnet assembly 600 may be supported by a ferromagnetic frame. FIG. 8 illustrates an example of an apparatus 800 including a C-shaped yoke 820 configured to support the bi-planar magnet assembly 600 of FIG. 6, in accordance with some embodiments of the technology described herein. Such C-shaped yokes 820 may be formed of ferromagnetic material (e.g., steel, silicon steel, CoFe, etc.) to direct magnetic flux produced by the magnet assembly 600 and increase B₀ magnetic field efficiency. Aspects of C-shaped ferromagnetic yokes 820 are described in U.S. Pat. No. 10,353,030, granted on Jul. 16, 2019, filed on Sep. 13, 2019, and titled “Low-Field Magnetic Resonance Imaging Methods and Apparatus,” which is incorporated by reference in its entirety herein.

In some embodiments, the magnet assembly 600 may be coupled to the ferromagnetic frame through a non-magnetic support (not shown). For example, non-ferromagnetic components (e.g., plastic components, fiberglass components) with a profile mirroring the profile of the magnet assembly 600 may house and support the magnet assembly 600. The non-ferromagnetic components may be coupled to the ferromagnetic frame.

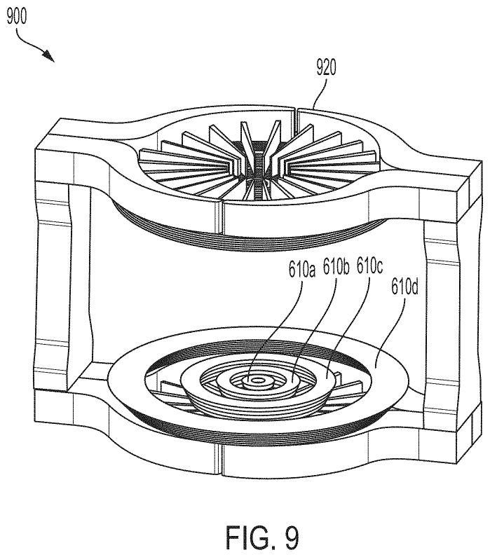

Alternatively, magnet assembly 600 may be incorporated into an apparatus including a symmetric frame structure, examples of which are shown in FIGS. 9-11, in accordance with some embodiments of the technology described herein. In the example of FIG. 9, symmetric frame structure 920 may include two or more posts to direct and concentrate the magnetic flux produced by the magnet assembly 600. By capturing magnetic flux and directing it to the region between B0 magnets 210, less permanent magnet material can be used in B0 magnets 210 to achieve a desired field strength, thus reducing the size, weight, and cost of the B0 magnet. Alternatively, for given permanent magnets, the field strength can be increased, thus improving the signal-to-noise ratio (SNR) of the system without having to use increased amounts of permanent magnet material.

In some embodiments, apparatus 900 includes blades 940 configured to enhance gradient magnetic fields generated by an MRI system that includes apparatus 900, in accordance with some embodiments of the technology described herein. Blades 940 may be arranged to cover the surface behind the gradient coils (not pictured) in a sparse manner, providing improved gradient field efficiency while minimizing eddy current conduction. In some embodiments, the blades 940 may be arranged in a radial manner, extending towards a common center in the collection area between the multi-pronged members of frame structure 920. Blades 940 may not meet or touch the common center in order to prevent the formation of a conduction path for eddy currents between opposing blades 940. As a result, the eddy current time constants for exemplary apparatus 900 may be less than half the eddy current time constants for comparable C-shaped designs.

In some embodiments, to provide improved gradient field efficiency, blades 940 may be formed of a ferromagnetic material. The blades may be formed of, for example, low carbon steel, CoFe, and/or silicon steel to provide the desired magnetic properties. The blades 940 may be formed of a same ferromagnetic material as frame structure 920, or may be formed of a different ferromagnetic material as frame structure 920.

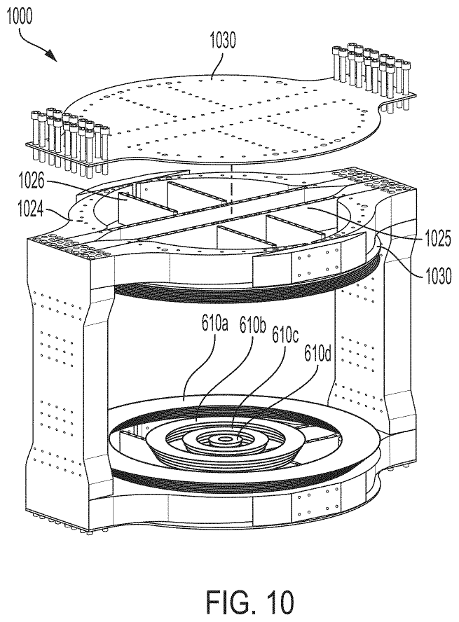

As shown in the example of FIG. 10, magnet assembly 600 may be incorporated into an apparatus 1000 comprising a frame 1020 having an alternative arrangement of blades 1026, in accordance with some embodiments of the technology described herein. In some embodiments, frame 1020 includes posts coupled to multi-pronged members. Frame 1020 also may include one or more connectors 1025 extending between opposite ends of posts 1022. The connectors 1025 may secure the posts to one another, increasing structural rigidity. In some embodiments, the connectors 1025 may be substantially parallel to one of the x- or y-gradient fields, providing additional improvement to the gradient field efficiency in that direction.

In some embodiments, apparatus 1000 may include blades 1026. Blades 1026 may be similar to blades 940 of apparatus 900. Blades 1026, however, may be arranged substantially parallel to a direction of one of the other gradient fields (e.g., one of the x- or y-gradient fields) rather than in a radial arrangement as in apparatus 900. Blades 1026 may be arranged substantially parallel to a direction of one of the gradient fields to provide improved gradient field efficiency during operation of the MRI system.

In some embodiments, the apparatus 1000 may include one or more non-conductive supports 1030 configured to cover the components of the frame 1020 and provide support to B₀ magnets 610a-d and blades 1026. In some embodiments, structural foam may be inserted into the spaces between the non-conductive supports 1030, the frame 1020, connectors 1025, and/or blades 1026. The non-conductive supports 1030 may be formed of a non-conductive laminate material such as G-10.

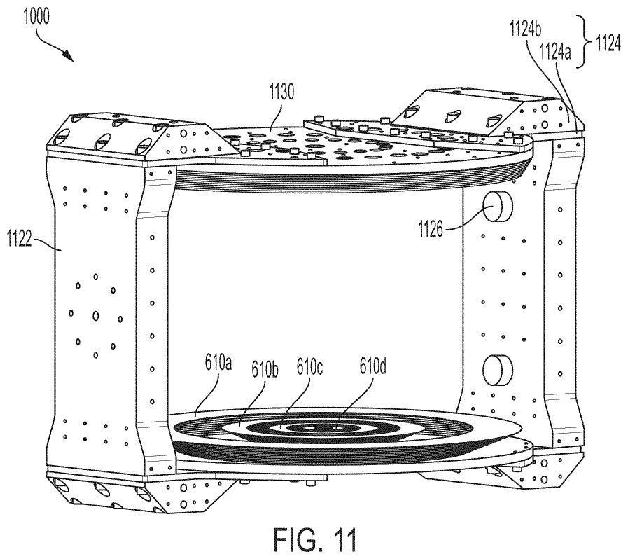

As shown in the example of FIG. 11, magnet assembly 600 may be incorporated into an apparatus 1100 including posts 112 secured to plates 1130 by connection assemblies 1124, in accordance with some embodiments of the technology described herein. In some embodiments, the connection assemblies 1124 may include a first connector 1124a and a second connector 1124b. The first connector 1124a may connect one of the posts 1122 to one of the plates 1130. For example, and as shown in FIG. 11, the first connector 1124a may be a substantially planar plate extending over the plate 1130 so that fasteners may extend through the first connector 1124a and secure the first connector 1124a to the plate 1130. First connector 1124a may be secured to the post 1122 by additional fasteners extending through the second connector 1124b, the first connector 1124a, and the post 1122. Forming the connection assembly 1124 out of multiple “layered” components may reduce manufacturing costs (e.g., by simplifying machining processes) and/or reduce magnetic saturation effects within the apparatus 1100.

In some embodiments, the second connector 1124b may be configured to increase the magnetic flux capacity of the apparatus 1100. For example, the second connector 1124b may have a wedge-like shape as shown in the examples of FIG. 11 to direct and concentrate magnetic flux from the posts 1122 back into the imaging region between the B₀ magnets 610a-d.

In some embodiments, plates 1130 may be configured to support B₀ magnets 610a-d. Plates 1130 may be formed from solid ferromagnetic sheet material. In some embodiments, plates 1130 may include one or more holes to reduce the weight of the plates 1130 and/or to allow for cooling or venting of the apparatus 1100 during MR imaging.

In some embodiments, apparatus 1100 may include additional permanent magnets 1126 positioned on inward-facing surfaces of posts 1122. The permanent magnets 1126 may be positioned and/or shaped to reduce inhomogeneity of the B₀ magnetic field and may be used in addition to or as a replacement for shim coils and/or passive shims positioned adjacent the B₀ magnets 610a-d. In some embodiments, permanent magnets 1126 may be polarized along a direction perpendicular to a plane of the inward-facing surfaces of the posts 1122 (e.g., toward or away from a common center of the concentric B₀ permanent magnet rings 610a-d). In some embodiments having two permanent magnets 1126, each of the two permanent magnets 1126 may have opposing polarizations. For example, a first of the permanent magnets 1126 may have a polarization directed toward the inward-facing surfaces of the posts 1122 and a second of the permanent magnets 1126 may have a polarization direction away from the inward-facing surfaces of the posts 1122. It should also be appreciated that permanent magnets 1126 may be included in any of the embodiments described herein, including apparatuses 800, 900, and/or 1000 described herein.

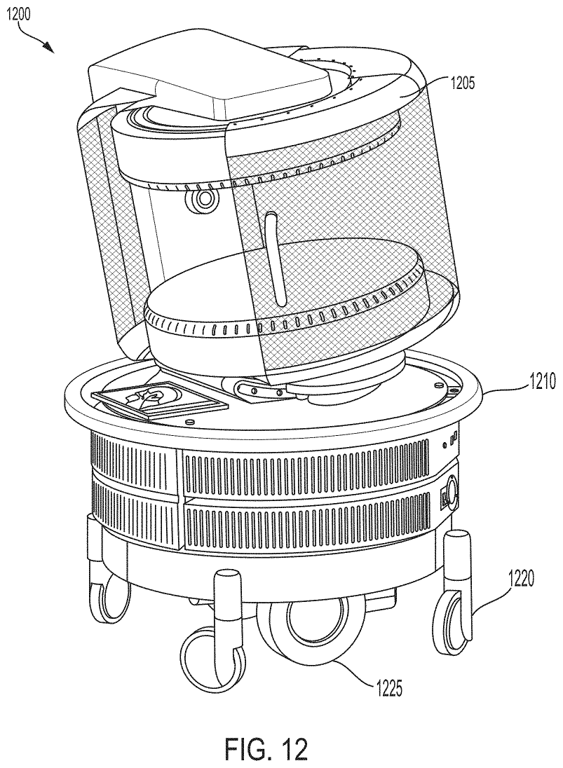

Using the techniques described herein, the inventors have developed portable, low power MRI systems capable of being brought to the patient, providing affordable and widely deployable MRI where it is needed. FIG. 12 shows an example of a portable, low-field MRI system 1000 including the magnet assembly 800 of FIG. 8, in accordance with some embodiments of the technology described herein. The magnet assembly 800 may be surrounded by a non-ferromagnetic housing 1205 and supported by a base 1210, as shown in the example of FIG. 12. Base 1210 may house the power components and/or electronics discussed in connection with FIG. 1, including power components configured to operate the MRI system 1200.

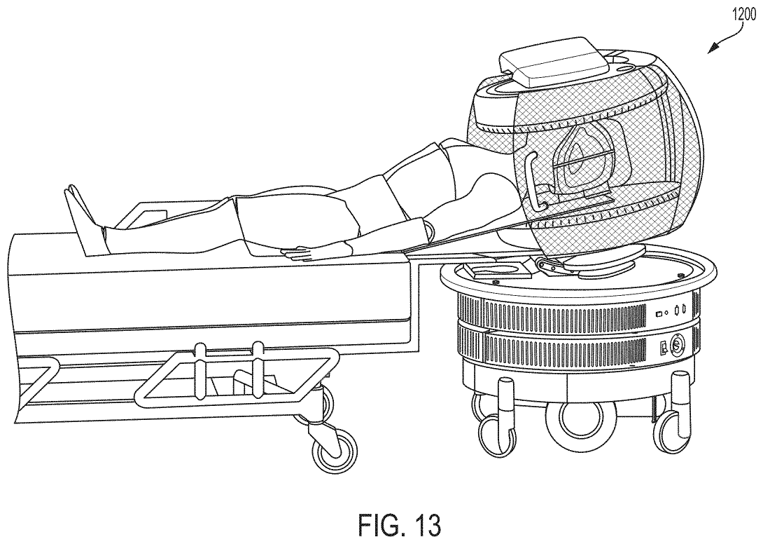

Base 1210 may also include one or more transport mechanisms 1220 which enable point-of-care use of MRI system 1200, in accordance with some embodiments of the technology described herein. In the example of FIG. 12, the transport mechanisms 1220 are depicted as wheels, but other transport mechanisms may be used. In some embodiments, transport mechanisms 1220 may include a motorized component 1225 may be provided to allow the MRI system 1200 to be driven from location to location, for example, using a control such as a joystick or other control mechanism provided on or remote from the MRI system 1000. In this manner, MRI system 1200 can be transported to the patient and maneuvered to the bedside to perform imaging, as illustrated in FIG. 13.

FIG. 13 depicts the use of the portable MRI system of FIG. 12 to perform a brain scan of a patient, in accordance with some embodiments of the technology described herein. During the brain scan, the MRI system 1200 may be used to capture at least one magnetic resonance image of the patient for clinical use.

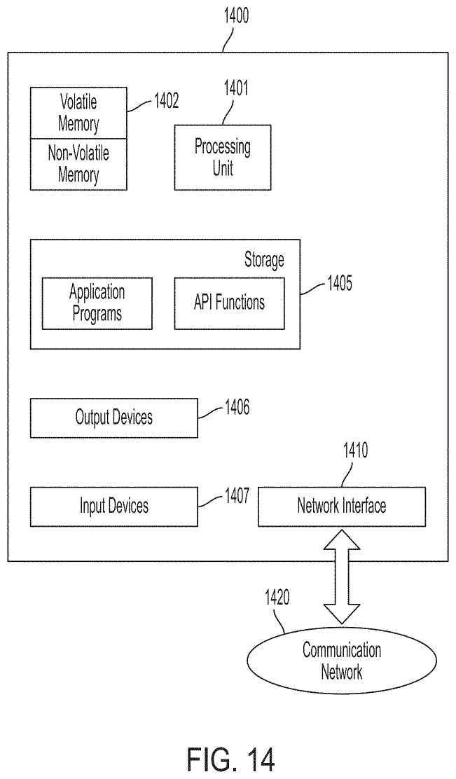

FIG. 14 shows, schematically, an illustrative computer 1400 on which any aspect of the present disclosure may be implemented.

In the embodiment shown in FIG. 14, the computer 1400 includes a processing unit 1401 having one or more processors and a non-transitory computer-readable storage medium 1402 that may include, for example, volatile and/or non-volatile memory. The memory 1402 may store one or more instructions to program the processing unit 1401 to perform any of the functions described herein. The computer 1400 may also include other types of non-transitory computer-readable medium, such as storage 1405 (e.g., one or more disk drives) in addition to the system memory 1402. The storage 1405 may also store one or more application programs and/or resources used by application programs (e.g., software libraries), which may be loaded into the memory 1402.

The computer 1400 may have one or more input devices and/or output devices, such as devices 1406 and 1407 illustrated in FIG. 14. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards and pointing devices, such as mice, touch pads, and digitizing tablets. As another example, the input devices 1407 may include a microphone for capturing audio signals, and the output devices 1406 may include a display screen for visually rendering, and/or a speaker for audibly rendering, recognized text. As another example, the input devices 1407 may include sensors (e.g., electrodes in a pacemaker), and the output devices 1406 may include a device configured to interpret and/or render signals collected by the sensors (e.g., a device configured to generate an electrocardiogram based on signals collected by the electrodes in the pacemaker).

As shown in FIG. 14, the computer 1400 may also comprise one or more network interfaces (e.g., the network interface 1410) to enable communication via various networks (e.g., the network 1420). Examples of networks include a local area network or a wide area network, such as an enterprise network or the Internet. Such networks may be based on any suitable technology and may operate according to any suitable protocol and may include wireless networks, wired networks or fiber optic networks. Such networks may include analog and/or digital networks.

Having thus described several aspects of at least one embodiment of this technology, it is to be appreciated that various alterations, modifications, and improvements will readily occur to those skilled in the art.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semi-custom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, the various methods or processes outlined herein may be coded as software that is executable on one or more processors running any one of a variety of operating systems or platforms. Such software may be written using any of a number of suitable programming languages and/or programming tools, including scripting languages and/or scripting tools. In some instances, such software may be compiled as executable machine language code or intermediate code that is executed on a framework or virtual machine. Additionally, or alternatively, such software may be interpreted.

The techniques disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more processors, perform methods that implement the various embodiments of the present disclosure described above. The computer-readable medium or media may be transportable, such that the program or programs stored thereon may be loaded onto one or more different computers or other processors to implement various aspects of the present disclosure as described above.

The terms “program” or “software” are used herein to refer to any type of computer code or set of computer-executable instructions that may be employed to program one or more processors to implement various aspects of the present disclosure as described above. Moreover, it should be appreciated that according to one aspect of this embodiment, one or more computer programs that, when executed, perform methods of the present disclosure need not reside on a single computer or processor, but may be distributed in a modular fashion amongst a number of different computers or processors to implement various aspects of the present disclosure.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The terms “approximately” and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

What is claimed is:

1. An apparatus for providing a B₀ magnetic field for a magnetic resonance imaging (MRI) system, the apparatus comprising:
a cylindrical shell forming a bore extending along a common longitudinal direction, the cylindrical shell comprising:
a first plurality of ferromagnetic rings including a first ferromagnetic ring with an angularly varying magnetization orientation; and
a second plurality of rings.

2. The apparatus of claim 1, wherein the first ferromagnetic ring is manufactured by swaging.

3. The apparatus of claim 1, wherein the second plurality of rings comprises one or more ferromagnetic rings with an angularly varying magnetization orientation.

4. The apparatus of claim 3, wherein the second plurality of rings comprises a second ferromagnetic ring with an angularly varying magnetization orientation, wherein the magnetization orientations of the first and second ferromagnetic rings angularly vary in opposing directions.

5. The apparatus of claim 1, wherein the second plurality of rings comprises one or more non-ferromagnetic rings.

6. The apparatus of claim 1, wherein the first plurality of ferromagnetic rings are interspersed with the second plurality of rings.

7. The apparatus of claim 1, wherein each of the first plurality of ferromagnetic rings and each the second plurality of rings have a same diameter to provide a bore having a constant diameter along the common longitudinal direction.

8. The apparatus of claim 1, wherein at least two of the plurality of ferromagnetic rings have different inner diameters.

9. The apparatus of claim 1, wherein the first plurality of ferromagnetic rings and second plurality of rings have different diameters so that a diameter of the bore is larger at one end of the bore than at another end of the bore.

10. The apparatus of claim 1, wherein the B₀ magnetic field has a field strength that is greater than 0.02 T and less than 0.2 T.

11. The apparatus of claim 1, wherein the B₀ magnetic field has a field strength that is greater than 0.05 T and less than 0.1 T.

12. The apparatus of claim 1, wherein the B₀ magnetic field has a homogeneity of substantially 10 ppm within an imaging region disposed within the bore.

13. The apparatus of claim 1, wherein the B₀ magnetic field has a homogeneity less than or equal to substantially 1000 ppm within an imaging region disposed within the bore.

14. The apparatus of claim 1, wherein all ferromagnetic material in the apparatus combined weighs less than 400 kg.

15. The apparatus of claim 1, wherein all ferromagnetic material in the apparatus combined weighs less than 40 kg.

16. A magnetic resonance imaging (MRI) system, comprising:
the apparatus of claim 1;
a plurality of gradient coils configured to, when operated, generate magnetic fields to provide spatial encoding of emitted magnetic resonance signals;
at least one radio frequency transmit coil; and
a power system configured to provide power to the gradient coils and the at least one radio frequency transmit coil.

17. A method of manufacturing an apparatus for providing a B₀ magnetic field for a magnetic resonance imaging (MRI) system, the method comprising:
manufacturing a ferromagnetic cylindrical shell at least in part by:
placing a magnetic metal alloy powder in an annular volume between an outer cylindrical tube and an inner cylindrical tube;
applying a magnetic field to the magnetic metal alloy powder while compressing the magnetic metal alloy powder;
bonding the magnetic metal alloy powder to form at least a part of the ferromagnetic cylindrical shell;
magnetizing the ferromagnetic cylindrical shell to have an angularly varying magnetization orientation;
partitioning the ferromagnetic cylindrical shell into a first plurality of ferromagnetic rings; and
assembling, from the first plurality of ferromagnetic rings and a second plurality of rings, a cylindrical shell forming a bore extending along a common longitudinal direction.

18. The method of claim 17, wherein the second plurality of ferromagnetic rings comprises one or more ferromagnetic rings with an angularly varying magnetization orientation.

19. The method of claim 18, wherein the first plurality of ferromagnetic rings comprises a first ferromagnetic ring with an angularly varying magnetization orientation, wherein the second plurality of rings comprises a second ferromagnetic ring with an angularly varying magnetization orientation, wherein the magnetization orientations of the first and second ferromagnetic rings angularly vary in opposing directions.

20. The method of claim 17, wherein the second plurality of rings comprises one or more non-ferromagnetic rings.

21. The method of claim 17, wherein the assembling comprising using the second plurality of rings as spacers among rings in the first plurality of ferromagnetic rings so that the first plurality of ferromagnetic rings are interspersed with the second plurality of rings.

22. A method of manufacturing an apparatus for providing a B₀ magnetic field for a magnetic resonance imaging (MRI) system, the method comprising:
manufacturing a ferromagnetic cylindrical shell at least in part by:
placing magnetic metal alloy powder and non-ferromagnetic powder in an annular volume between an outer cylindrical tube and an inner cylindrical tube;
applying a magnetic field to the magnetic metal alloy powder while compressing the magnetic metal alloy powder;
bonding the magnetic metal alloy powder and the non-ferromagnetic powder to form the ferromagnetic cylindrical shell; and
magnetizing the ferromagnetic cylindrical shell to have an angularly varying magnetization orientation.

23. The method of claim 22, wherein placing the non-magnetic powder between the two cylindrical tubes comprises interspersing the non-ferromagnetic powder with the magnetic metal alloy powder.

24. The method of claim 22, further comprising at least partially removing the two cylindrical tubes from the ferromagnetic cylindrical shell.

25. A method of manufacturing an apparatus for providing a B₀ magnetic field for a magnetic resonance imaging (MRI) system, the method comprising:
manufacturing a ferromagnetic cylindrical shell at least in part by:
placing magnetic metal alloy powder in an annular volume between an outer cylindrical tube and an inner cylindrical tube;
applying a magnetic field to the magnetic metal alloy powder while compressing the magnetic metal alloy powder;
bonding the magnetic metal alloy powder to form the ferromagnetic cylindrical shell;
selectively magnetizing first ring regions of the ferromagnetic cylindrical shell to have a first angularly varying magnetization orientation; and
selectively magnetizing second ring regions of the ferromagnetic cylindrical shell to have a second angularly varying magnetization orientation, the second angularly varying magnetization orientation varying in a direction opposing that of the first angularly varying magnetization orientation.

26. The method of claim 25, wherein the first ring regions are interspersed with the second ring regions.

27. The method of claim 25, further comprising at least partially removing the two cylindrical tubes from the ferromagnetic cylindrical shell.
